(19) United States
(12) Patent Application Publication
(10) Pub. No.: US 2022/0313641 A1
(43) Pub. Date: Oct. 6, 2022

(54) SPREADABLE NUTRITIONAL SUPPLEMENT AND SPREADABLE MEDICINE

(71) Applicant: LeteBara L.L.C., Gettysburg, PA (US)

(72) Inventors: Yianni George Barakos, Gettysburg, PA (US); Philip George Letendre, Gettysburg, PA (US)

(21) Appl. No.: 17/713,843

(22) Filed: Apr. 5, 2022

Related U.S. Application Data
(60) Provisional application No. 63/200,963, filed on Apr. 6, 2021.

(51) Int. Cl. A61K 31/198; A61K 31/522; A61K 31/14; A61K 31/685; A61K 9/00; A61K 31/519; A61K 31/573; A61K 31/5415; A61K 31/192; A61K 31/635; A61K 31/196; A61K 31/05; A61K 31/4985; A61K 31/585; A61K 31/167; A61K 31/485; A61K 31/137; A61K 31/138; A61K 47/46; A23L 33/175; A23L 33/15; A23L 25/10; A23L 21/10; A23D 7/005

(52) U.S. Cl. CPC A61K 31/198; A61K 31/522; A61K 31/14; A61K 31/685; A61K 9/0056; A61K 31/519; A61K 31/573; A61K 31/5415; A61K 31/192; A61K 31/635; A61K 31/196; A61K 31/05; A61K 31/4985; A61K 31/585; A61K 31/167; A61K 31/485; A61K 31/137; A61K 31/138; A61K 47/46; A23L 33/175; A23L 33/15; A23L 25/10; A23L 21/10; A23D 7/0056; A23V 2002/00

(57) ABSTRACT

A supplemental spread including at least one of block chain amino acids (“BCAAs”), essential amino acids (“EAAs”), creatine, caffeine, taurine, tyrosine, alpha glycerylphosphorylcholine (alpha-GPC), or combinations thereof. The supplemental spread can have a viscosity ranging from about 450 centipoise to about 250,000 centipoise at a temperature ranging from about 20° C. to about 25° C.

### CROSS-REFERENCE TO RELATED APPLICATIONS

The present application claims priority to and benefit of U.S. Provisional Patent Application No. 63/200,963, filed Apr. 6, 2021, and entitled “Spreadable Nutritional Supplement and Spreadable Medicine,” the entirety of which is herein incorporated by reference.

### BACKGROUND

Supplements can be added to provide nutrients that may otherwise not be consumed in sufficient quantities. One option for consuming supplements includes taking a pill with liquid. Another option is to add powder to a liquid in a container such as a cup or a shaker.

### SUMMARY

The present disclosure relates to a supplemental spread with nutritional ingredients, exercise enhancement ingredients, and/or medicine(s) (e.g., over-the-counter medicine(s) and/or prescription medicine(s)).

Supplements may be used for exercise and/or diet enhancement purposes. Medicinal dosages may be used for managing the diagnosis, prognosis, prevention, treatment or palliation of injury or disease.

Exercise or “pre-workout” supplements are currently limited to messy powders mixed into drinks; pre-made shakes; and pills. These are popular delivery methods but are not always ideal. Pills may be difficult to swallow. Gummies may be difficult to chew, inconveniently get stuck to teeth, or cause problems for those with dental issues. Supplements are typically consumed separate from other foods. It is difficult to effectively spread a liquid or pill on existing foods, such as bread or fruit. Thus, there exists a need for a supplement, such as a supplement with an exercise enhancing ingredient available in a spreadable form, such as peanut butter or jam.

Supplements that are nutritional such as vitamins and other micronutrients, e.g., branch chain amino acids (“BCAAs”), and essential amino acids (“EAAs”), may be delivered in pill, gummy, or powder form.

Medicinal drug dosage delivery methods have limited delivery methods including pills, liquids, vapors, and lotions. The delivery methods by mouth can be problematic as pills and capsules may be difficult to swallow, particularly for the elderly community. Liquid medicines may be messy.

Thus, there exists a need for a supplemental spread that may be consumed with foods and without the need for a liquid, pill, powder, or gummy. The supplemental spread can be delivered in a consumable spreadable form, such as peanut butter or jam.

The present disclosure provides a supplemental spread that can be used for exercise and/or diet enhancement purposes as well as for managing the diagnosis, prognosis, prevention, treatment or palliation of injury or disease.

In some aspects, the techniques described herein relate to a supplemental spread including: at least one of block chain amino acids (“BCAAs”), essential amino acids (“EAAs”), creatine, caffeine, taurine, tyrosine, alpha glycerylphosphorylcholine (alpha-GPC), or combinations thereof, wherein the supplemental spread has a viscosity ranging from about 450 centipoise to about 250,000 centipoise at a temperature ranging from about 20° C. to about 25° C.

In some aspects, the techniques described herein relate to a supplemental spread, wherein the supplemental spread includes a spreadable form and is configured to be spread on a surface via a tool, whereby the supplemental spread sticks to the surface while being spread across the surface.

In some aspects, the techniques described herein relate to a supplemental spread, further including vitamin(s).

In some aspects, the techniques described herein relate to a supplemental spread, further including an over-the-counter medicine(s) and/or a prescription medicine(s).

In some aspects, the techniques described herein relate to a supplemental spread, wherein the prescription medicine(s) include at least one of tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

In some aspects, the techniques described herein relate to a supplemental spread, wherein the over-the-counter medicine(s) include at least one of Ibuprofen, acetaminophen, dextromethorphan, pseudoephedrine, dimenhydrinate, diphenhydramine, or combinations thereof.

In some aspects, the techniques described herein relate to a supplemental spread, wherein the supplemental spread is absent a protein source.

In some aspects, the techniques described herein relate to a supplemental spread, wherein the supplemental spread is configured to be consumed on a stand-alone basis.

In some aspects, the techniques described herein relate to a supplemental spread, wherein the supplemental spread is embedded in peanut butter, jam, or butter.

In some aspects, the techniques described herein relate to a method of using a supplemental spread including: spreading the supplemental spread on a surface via a tool, whereby the supplemental spread sticks to the surface while being spread across the surface, wherein the supplemental spread includes at least one of block chain amino acids (“BCAAs”), essential amino acids (“EAAs”), creatine, caffeine, taurine, tyrosine, alpha glycerylphosphorylcholine (alpha-GPC), or combinations thereof, and wherein the supplemental spread has a viscosity ranging from about 450 centipoise to about 250,000 centipoise at a temperature ranging from about 20° C. to about 25° C.

In some aspects, the techniques described herein relate to a method, wherein the supplemental spread further includes vitamin(s).

In some aspects, the techniques described herein relate to a method, wherein the supplemental spread further includes an over-the-counter medicine(s) and/or a prescription medicine(s).

In some aspects, the techniques described herein relate to a method, wherein the prescription medicine(s) include tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

In some aspects, the techniques described herein relate to a method, wherein the over-the-counter medicine(s) include at least one of Ibuprofen, acetaminophen, dextromethorphan, pseudoephedrine, dimenhydrinate, diphenhydramine, or combinations thereof.

In some aspects, the techniques described herein relate to a method, wherein the supplemental spread is absent a protein source.

### DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 depicts a schematic of a method of using a supplemental spread according to some embodiments of the present disclosure.

FIG. 2 depicts a flow chart of a method of using a supplemental spread according to some embodiments of the present disclosure.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

All prior patents and publications referenced herein are incorporated by reference in their entireties.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “between” does not necessarily require being disposed directly next to other elements. Generally, this term means a configuration where something is sandwiched by two or more other things. At the same time, the term “between” can describe something that is directly next to two opposing things. Accordingly, in any one or more of the embodiments disclosed herein, a particular structural component being disposed between two other structural elements can be:
disposed directly between both of the two other structural elements such that the particular structural component is in direct contact with both of the two other structural elements;
disposed directly next to only one of the two other structural elements such that the particular structural component is in direct contact with only one of the two other structural elements;
disposed indirectly next to only one of the two other structural elements such that the particular structural component is not in direct contact with only one of the two other structural elements, and there is another element which juxtaposes the particular structural component and the one of the two other structural elements;
disposed indirectly between both of the two other structural elements such that the particular structural component is not in direct contact with both of the two other structural elements, and other features can be disposed therebetween; or
any combination(s) thereof.

As used herein “embedded” means that a first material is distributed throughout a second material.

The present disclosure provides a supplemental spread as an exercise enhancing, diet enhancing, and/or medicinal spread comprising a mixture of supplements and/or medicines with spreadable ingredients.

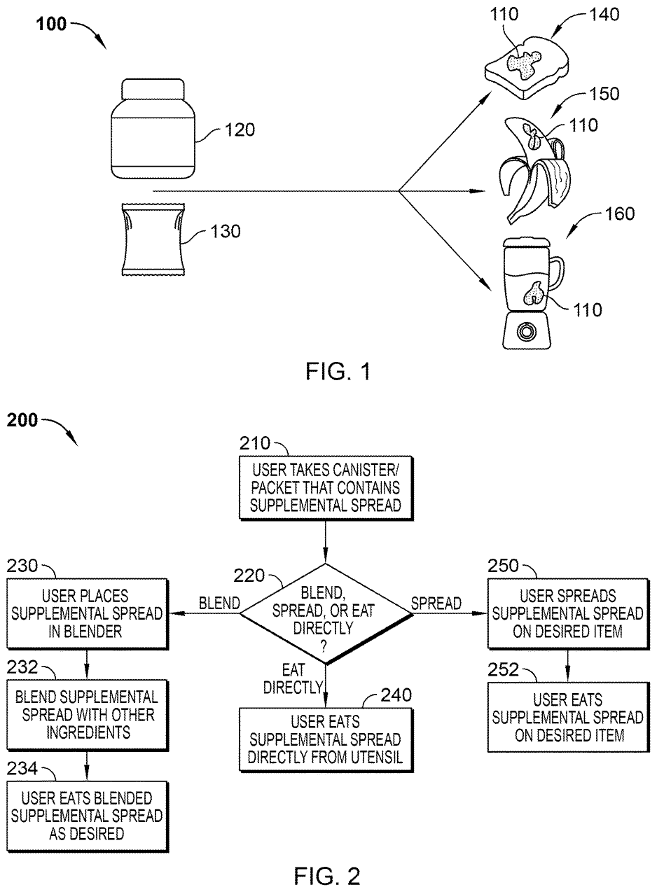

FIG. 1 depicts a schematic of a method 100 of using a supplemental spread 110 according to some embodiments of the present disclosure. FIG. 1 shows a canister 120 and a packet 130 that can contain the supplemental spread 110. The supplemental spread 110 from the canister 120 and the packet 130 can be transferred to a bread-based item 140, fruit 150, and/or a blender 160. Although FIG. 1 shows the bread-based item 140 as bread, other food items can be used (e.g., a cracker, cookie, biscuit, bar, bagel, muffin, roll, doughnut, or cake). Although fruit 150 is shown as a banana, other fruits can be used (e.g., apple, pear, orange, grapefruit, mandarin, lime, nectarine, apricot, peach, plum, mango, strawberry, raspberry, blueberry, kiwifruit, passionfruit, watermelon, rock melon, honeydew melon, tomato or avocado). Besides bread-based item 140 (e.g., bread-based items) and fruit 150, other food items can be used too. For example, the supplemental spread 110 can be placed on vegetables and legumes.

In some embodiments, the supplemental spread 110 from the canister 120 and/or the packet 130 can be placed in the blender 160. Various foods and liquids can be added along with the supplemental spread 110 to the blender 160. The supplemental spread 110 can be blended into a smoothie or shake prior to ingestion. The supplemental spread 110 can provide benefits to a user including exercise enhancements, diet enhancements, and/or medicinal enhancements/benefits.

A viscosity of the supplemental spread 110 can range from about 500 centipoise (similar to jelly) to about 250,000 centipoise (similar to peanut butter) or any intervening value or range. For example, in some embodiments, the viscosity of the supplemental spread 110 can range from about 500 centipoise to about 25,000 centipoise, from about 500 centipoise to about 50,000 centipoise, from about 500 centipoise to about 75,000 centipoise, from about 500 centipoise to about 100,000 centipoise, from about 500 centipoise to about 125,000 centipoise, from about 500 centipoise to about 150,000 centipoise, from about 500 centipoise to about 175,000 centipoise, from about 500 centipoise to about 200,000 centipoise, or from about 500 centipoise to about 225,000 centipoise. In some embodiments, the viscosity of the supplemental spread 110 can range from about 25,000 centipoise to about 250,000 centipoise, from about 50,000 centipoise to about 250,000 centipoise, from about 75,000 centipoise to about 250,000 centipoise, from about 100,000 centipoise to about 250,000 centipoise, from about 125,000 centipoise to about 250,000 centipoise, from about 150,000 centipoise to about 250,000 centipoise, from about 175,000 centipoise to about 250,000 centipoise, from about 200,000 centipoise to about 250,000 centipoise, from about 225,000 centipoise to about 250,000 centipoise. In some embodiments, the viscosity of the supplemental spread 110 can range from about 75,000 centipoise to about 150,000 centipoise, from about 50,000 centipoise to about 125,000 centipoise, from about 175,000 centipoise to about 225,000 centipoise, from about 50,000 centipoise to about 225,000 centipoise, from about 75,000 centipoise to about 150,000 centipoise, from about 100,000 centipoise to about 200,000 centipoise, or any other intervening range between about 500 centipoise to about 250,000 centipoise. In some embodiments, the supplemental spread 110 can have a viscosity greater than about 500 centipoise, greater than about 25,000 centipoise, greater than about 50,000 centipoise, greater than about 75,000 centipoise, greater than about 100,000 centipoise, greater than about 125,000 centipoise, greater than about 150,000 centipoise, greater than about 175,000 centipoise, greater than about 200,000 centipoise, or greater than about 225,000 centipoise. In some embodiments, the supplemental spread 110 can have a viscosity less than about 25,000 centipoise, less than about 50,000 centipoise, less than about 75,000 centipoise, less than about 100,000 centipoise, less than about 125,000 centipoise, less than about 150,000 centipoise, less than about 175,000 centipoise, less than about 200,000 centipoise, less than about 225,000 centipoise, or less than about 250,000 centipoise.

The viscosity of the supplemental spread 110 is measured at room temperature (e.g., a temperature ranging from about 20° C. to about 25° C.). Similar to peanut butter or jelly, the supplemental spread 110 with remain consistent unless heated beyond the temperature range of about 20° C. to about 25° C.

In some embodiments, the ingredients of the supplemental spread 110 can be pre-mixed in a spreadable form for immediate consumption. The ingredients of the supplemental spread 110 may be combined with a variety of spread ingredients including, but not limited to, peanut butter, jelly, jam, butter, and combinations thereof.

The supplemental spread 110 is mixed at production with the purpose of consistency throughout the spread. As a result, a dosage of “1 tablespoon”, for example, will be consistent and the dosages of additives will be consistent throughout. As a result, a “suggested use” for supplement/vitamin purposes may give an exact dosage (tablespoon, teaspoon, etc.). The suggested use will depend on the maximum daily amount of ingredients as well as the intended range for that ingredient. For example, vitamin D can have an upper limit (i.e., maximum daily amount) of about 50 mcg per day. The suggested use may correspond to an amount of the supplemental spread 110 that will deliver the maximum daily amount, e.g., 50 mcg of vitamin D per day. Alternatively, the suggested use may correspond to providing an amount less than the daily maximum amount (e.g., less than 90%, less than 80%, less than 70%, less than 60%, less than 50%, less than 40%, less than 30%, less than 20%, or less than 10%). For example, the suggested use may correspond to half a daily maximum amount of vitamin D (e.g., 25 mcg of vitamin D per day).

A method of making the supplemental spread 110 may include measuring appropriate dosages of the ingredients (e.g., creatine, caffeine, beta-alanine, taurine, amino acids, other ingredients as described herein, as well as other ingredients not listed) and mixing the ingredients with a base ingredient such as but not limited to peanut butter, jelly, jam, or butter to create a spreadable-form of the ingredients.

Shelf life of the supplemental spread 110 will depend on the lowest shelf life of any ingredient. For example, if the lowest shelf life of the ingredients is 1 year, the supplemental spread 110 will have a shelf life of 1 year. Traditional ingredients of supplements can have a life of 2 years unopened. BCAAs or other amino acids (if added to the supplemental spread 110) may shorten the shelf life of the supplemental spread 110 because they have a shorter shelf life than 2 years and begin to break down with age. The shelf life of the supplemental spread 110 can be modified by what ingredients are added. Some peanut butter has an unopened shelf life of 12-24 months unopened and 2-3 months opened. Some jelly has an unopened shelf life of at least 12 months and at least 6 months opened and refrigerated. The United States Department of Agriculture (USDA) guidelines state that jelly or jam can be stored unopened in the pantry for up to 12 months. However, some homemade preserves canned in a boiling water bath can be stored in a cool dark place for up to two years. Once opened, jam should be refrigerated and stored for up to three months and jelly for up to six months. Source: USDA Website. From our research, USDA recommendations are conservative compared to other recommendations which are included here:

In some embodiments, the shelf life of the supplemental spread 110 is greater than 1 year, greater than 2 year, greater than 3 years, greater than 4 years, less than 5 years, less than 4 years, less than 3 years, from about 1 year to about 5 years, or any intervening range such as from about 2 years to about 3 years or from about 1 year to about 4 years.

Table 1 below shows shelf life for unopened items as well as opened items in a refrigerator.

TABLE 1

| | | Opened Shelf Life in |
|---|---|---|
| Type of Preserve | Unopened Shelf Life | Fridge |
| Jam | Up to 2 years | 6 months to 1 year |
| Jelly | Up to 2 years | 6 months to 1 year |
| Freezer Jam | Up to 1 year (In | Up to 1 month |
| | Freezer) | |
| Low Sugar Jam/Jelly | Not recommended | 8-9 months |
| Sugar-Free Jam/Jelly | Not recommended | 6-9 months |
| Fruit Butter | Up to 2 years | 2-3 months |
| Marmalade | Up to 2 years | 6 months to 1 year |
| Conserves | Up to 2 years | 2-3 months |
| Chutney | Up to 2 years | 2-3 months |

The supplemental spread 110 can include a variety of ingredients. In some embodiments, the supplemental spread 110 can include ingredients like medicinal drug(s) such as over-the-counter medicine(s) and/or prescription medicines. Medicinal drug(s) can be used for managing the diagnosis, prognosis, prevention, treatment or palliation of injury or disease.

The medicinal drugs can include prescription medicines such as tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, and tadalafil.

The over-the-counter medicine(s) include, for example, ibuprofen, acetaminophen, dextromethorphan, pseudoephedrine, dimenhydrinate, and diphenhydramine. The over-the-counter medicine(s) can include pain relievers, cough suppressants, and antihistamines. The medicinal drugs can be used to provide benefits for the user as indicated by the medicinal drug.

A dose refers to a specific amount of ingredient (e.g., a medication) in the supplemental spread 110 taken at one time. In some embodiments, the supplemental spread 110 dosage concentration can be consistent with pill-form dosages.

The supplemental spread 110 can include ibuprofen as an ingredient. Ibuprofen is a nonsteroidal anti-inflammatory drug (NSAID) and works by reducing hormones that cause inflammation and pain in the body. Ibuprofen can be used to reduce fever and treat pain or inflammation caused by many conditions such as headache, toothache, back pain, arthritis, menstrual cramps, or minor injury. For some individuals, the maximum amount of ibuprofen for adults can be 800 milligrams per dose or 3200 mg per day (4 maximum doses). In some embodiments, for a single serving packet 130, the maximum amount of ibuprofen in the supplemental spread 110 for adults is 800 milligrams. In some embodiments, the canister 120 can include a plurality of servings. The amount of ibuprofen can increase (e.g., proportionally) along with an increase in the number of serving portions.

The supplemental spread 110 can include acetaminophen as an ingredient. Acetaminophen can be used to treat mild to moderate pain (from headaches, menstrual periods, toothaches, backaches, osteoarthritis, or cold/flu aches and pains) and to reduce fever. For some individuals, the maximum amount of acetaminophen is not to exceed a cumulative dose of 3.25 grams/day of acetaminophen. In some embodiments, for a single serving packet 130, the maximum amount of acetaminophen in the supplemental spread 110 for adults is 3.25 grams. In some embodiments, the canister 120 can include a plurality of servings. The amount of acetaminophen can increase (e.g., proportionally) along with an increase in the number of serving portions.

The supplemental spread 110 can include dextromethorphan as an ingredient. Dextromethorphan is a cough suppressant. It affects the signals in the brain that trigger cough reflex. Dextromethorphan can be used to treat a cough. Dextromethorphan is in a class of medications called antitussives. Dextromethorphan works by decreasing activity in the part of the brain that causes coughing. For lozenge dosage form, adults and children 12 years of age and older-5 to 15 mg every two to four hours, as needed; children 6 to 12 years of age—5 to 15 mg every two to six hours, as needed; children 4 to 6 years of age—5 mg every four hours, as needed; and children and infants up to 4 years of age—use is not recommended. For syrup dosage form: adults and children 12 years of age and older—30 mg every six to eight hours, as needed; children 6 to 12 years of age—7 mg every four hours or 15 mg every six to eight hours, as needed; children 4 to 6 years of age—3.5 mg every four hours or 7.5 mg every six to eight hours, as needed; children and infants up to 4 years of age—use is not recommended. For extended-release oral suspension dosage form: adults and children 12 years of age and older-60 mg every twelve hours, as needed; children 6 to 12 years of age—30 mg every twelve hours, as needed; children 4 to 6 years of age—15 mg every twelve hours, as needed; and children and infants up to 4 years of age—use is not recommended. In some embodiments, for a single serving packet 130, the maximum amount of dextromethorphan in the supplemental spread 110 for adults is based upon the dosage form as discussed herein. In some embodiments, the canister 120 can include a plurality of servings. The amount of dextromethorphan can increase (e.g., proportionally) along with an increase in the number of serving portions.

The supplemental spread 110 can include one or more vitamins (e.g., a composition of general vitamins) as ingredients. Vitamins can include, e.g., biotin (B7), choline, folate (B9), iron, magnesium, niacin (B3), pantothenic acid (B5), pyridoxine (B6), riboflavin (B2), thiamin (B1), cobalamin (B12), and vitamins A, C, D, E, and K.

The supplemental spread 110 can include one or more hormones such as melatonin, testosterone, or estrogen. Melatonin can help with the timing of circadian rhythms (24-hour internal clock) and with sleep. Testosterone helps maintain men's bone density, fat distribution, muscle strength and mass, facial and body hair, red blood cell production, sex drive, and sperm production. Estrogen contributes to cognitive health, bone health, the function of the cardiovascular system, and other essential bodily processes. Dosages for the hormones can range from about 1 mg to about 10 mg or higher. In some embodiments, recommended dosages are not to exceed 10 mg.

Vitamin A keeps your heart, lungs, liver and other organs working properly. Also called beta-carotene, vitamin A is important for reproductive, vision and immune system health. Vitamin A has an upper limit of 3,000 mcg (about 10,000 IU) per day.

There are eight different essential B vitamins—B1 (thiamin), B2 (riboflavin), B3 (niacin), B5 (pantothenic acid), B6 (pyridoxine), B7 (biotin), B9 (folate) and B12 (cobalamin). The essential B vitamins help convert carbohydrates, fats and proteins into energy. Several B vitamins are also necessary for cell development, growth and function. Thiamin (vitamin B1) does not have a known upper limit per day. Riboflavin (vitamin B2) does not have a known upper limit per day. Niacin (vitamin B3, nicotinic acid) has an upper limit of about 35 mg per day. Pantothenic acid (vitamin B5) does not have a known upper limit per day. Pyridoxine (vitamin B6, pyridoxal, pyridoxine, pyridoxamine) has an upper limit of about 35 mg per day. Biotin (vitamin B7) does not have a known upper limit per day. Folate (vitamin B9) has an upper limit of about 1,000 mcg per day. Cobalamin (vitamin B12) does not have a known upper limit per day.

Also known as ascorbic acid, vitamin C boosts the immune system and increases iron absorption from plant-based foods and supplements. Since vitamin C is an antioxidant, vitamin C protects cells from damaging free radicals. Vitamin C also aids in wound healing by helping the body produce collagen. Ascorbic acid (vitamin C) has an upper limit of about 2,000 mg per day.

Vitamin D builds strong bones by helping the body absorb calcium from food and supplements. Vitamin D also boosts the functioning of the immune system. Vitamin D has an upper limit of about 50 mcg (2,000 IU) per day.

Vitamin E protects cells from free radicals, boosts the immune system and helps prevent blood clots. Vitamin E has an upper limit of about 1,000 mg (nearly 1,500 IU natural vitamin E; 2,200 IU synthetic) per day.

Vitamin K is necessary for blood clotting and healthy bones. More vitamin K may be needed if a person has recently had bariatric surgery to lose weight or has a malabsorption disorder. Vitamin K does not have a known upper limit per day. The daily dose of vitamin K may range from about 60 mcg to about 120 mcg.

The supplemental spread 110 can include iron as an ingredient. Iron is a part of building red blood cells, specifically hemoglobin, a protein that bonds with oxygen to oxygen through the blood from the lungs to the cells throughout your body. Iron has an upper limit of about 45 mg per day.

The supplemental spread 110 can include magnesium as an ingredient. Magnesium plays an important role in the function of more than 300 enzymes that regulate various processes in the body, including muscle and nerve function, heart rhythms and glucose control. Magnesium has an upper limit of about 65 mg/day for children ages 1-3; 110 mg/day for children ages 4-8; and 350 mg/day for adults and children ages 9 and up.

The supplemental spread 110 can include zinc as an ingredient. Zinc is a mineral that plays an important role in immune function and is essential for normal growth and development during pregnancy and childhood. Iron has an upper limit of about 40 mg per day.

The supplemental spread 110 can include BCAAs as an ingredient. BCAAs are essential nutrients and are leucine, isoleucine, and valine. BCAAs stimulate the building of protein in muscle and possibly reduce muscle breakdown. The “branched-chain” refers to the chemical structure of these amino acids. A BCAA dosage for muscle enhancement is up to 20 grams a day taken in divided doses.

The supplemental spread 110 can include EAAs as an ingredient. EAAs include histidine isoleucine, leucine, lysine, methionine, phenylalanine, threonine, tryptophan, and valine. EAAs build vital proteins in the body that are used to create tissues, organs and muscles. EAAs also play a vital role in optimizing liver function and neurotransmitters in the brain. An EAA dosage for muscle enhancement is up to 20 grams a day taken in divided doses.

The supplemental spread 110 can include creatine as an ingredient. Creatine supplementation can improve athletic performance and increase muscle mass. Creatine can also be used to treat certain brain disorders, neuromuscular conditions, congestive heart failure and other conditions. In some examples, creatine has an upper limit of about 20 mg per day.

The supplemental spread 110 can include caffeine as an ingredient. Caffeine can improve a person's athletic performance if they take the correct amount at an appropriate time. Benefits include: better coordination, focus, and concentration, less pain and feelings of fatigue, and improved endurance in high intensity exercises. In some examples, caffeine has an upper limit of about 400 mg per day.

The supplemental spread 110 can include beta-alanine as an ingredient. Beta-alanine may help muscles perform for longer periods before they become fatigued. In some examples, beta-alanine has an upper limit of about 5 grams per day. Some daily doses range from about 2 grams to about 5 grams per day.

The supplemental spread 110 can include taurine as an ingredient. Taurine can enhance muscle contraction and delay muscle fatigue, which may benefit athletic performance. Taurine may increase fat burning during exercise to better fuel your performance. In some examples, taurine has an upper limit of about 6000 mg per day. Some daily doses range from about 500 to about 3000 mg per day.

The supplemental spread 110 can include tyrosine as an ingredient. Tyrosine is used in the body to make chemical messengers that are involved in conditions affecting the brain, such as mental alertness. Tyrosine can be used in protein supplements for an inherited disorder called phenylketonuria (PKU). Tyrosine may be used for alcohol use disorder, cocaine dependence, and memory and thinking skills. A daily dose range can be up to 150 mg/kg.

The supplemental spread 110 can include alpha glycerylphosphorylcholine (alpha-GPC) as an ingredient. Alpha-GPC administration can increase the release of the neurotransmitter acetylcholine and facilitates learning and memory. In athletes, alpha-GPC supplementation can prevent exercise-induced reductions in choline levels, increases endurance performance and growth hormone secretion. In some examples, a daily dose can range from about 3,000 mg to about 7,000 mg.

In some embodiments, the supplemental spread 110 is absent a protein source (e.g., no whey protein concentrate).

Depending on a desired dosage size of the supplemental spread 110, the ingredients, and the percentage of the ingredients relative to one another can vary. The suggested dosages will not exceed maximum daily dose of any given ingredient. For example, for some individuals, a maximum daily dose of caffeine is 400 mg. Therefore, a desired dosage is not to exceed 400 mg of caffeine per day.

In some embodiments, the supplemental spread 110 includes at least one of block chain amino acids (“BCAAs”), essential amino acids (“EAAs”), creatine, caffeine, taurine, tyrosine, alpha glycerylphosphorylcholine (alpha-GPC), or combinations thereof. Other ingredients as described herein can also added.

FIG. 2 depicts a flow chart of a method 200 of using a supplemental spread (e.g., the supplemental spread 110) according to some embodiments of the present disclosure. In method 200, a user (e.g., a consumer) takes the supplemental spread 110 from the canister 120 or the packet 130 in a first step 210 and makes a decision 220 whether to blend, spread, or eat directly. For example, a method of using the supplemental spread 110 may include consuming it as a stand-alone product or spreading the supplemental spread 110 on other foods such as bread or fruit in the appropriate quantity for a serving.

In some embodiments, the user can make the decision 220 to place the supplemental spread 110 in a blender (e.g., blender 160) 230. In some embodiments, a user can blend the supplemental spread 110 with other ingredients 232. The other ingredients can be liquids (e.g., water, orange juice, apple juice, milk, coconut water) and other foods (e.g., fruits as previously described, nuts, vegetables, legumes). After blending, the user eats the blended supplemental spread 110 as desired 234.

In some embodiments, the supplemental spread 110 can be consumed directly 240. A user (e.g., a consumer) can remove the supplemental spread 110 from the canister 120 and consume directly 240. For example, a tool (e.g., a utensil like a spoon, spatula, or knife) can be used to remove the supplemental spread 110 from the canister 120, and the supplemental spread 110 can be consumed directly 240 from the tool. The supplemental spread 110 from the packet 130 can also be dispersed onto a tool and then consumed directly 240 by the user. In some embodiments, a user can consume the supplemental spread 110 directly 240 from the canister 120 and/or the packet 130.

In some embodiments, the user can make a decision 220 to spread the supplemental spread 110 on a desired item 250. For example, the desired item can be a bread-based item 140 or fruit 150 as shown and described in relation to FIG. 1. As explained in the description of FIG. 1, the desired item can also be other items, such as a vegetable or legumes. A user can use a tool like a utensil (e.g., spoon, knife, or spatula) to spread the supplemental spread 110. The supplemental spread 110 can be spread on a surface via a tool, whereby the supplemental spread 110 sticks to the surface while being spread across the surface (e.g., a surface of the bread-based item 140). Once the supplemental spread 110 is on the desired item, a user can eat the supplemental spread 110 off the desired item or eat the supplemental spread 110 along with the desired item.

Spreads such as, but not limited to, peanut butter, jam, or butter are easily added to existing foods and recipes or can be consumed on a stand-alone basis. Similarly, the supplemental spread 110 can be added to existing foods and recipes or can be consumed on a stand-alone basis. The supplemental spread 110 does not need to be mixed with water and does not require a bottle. The supplemental spread 110 can be contained within the canister 120 and packet 130. The canister 120 and the packet 130 are easily transportable and do not require a shaker or pill swallowing for consumption.

The supplemental spread 110 can have the same composition regardless of its packaging. For example, the supplemental spread 110 can be the same whether the canister 120 or the packet 130 is used. The packet 130 can be used as a single-use/dose. For example, the supplemental spread 110 in the packet 130 can include a medical prescription that requires a precise amount of the prescription. The packet 130 can also serve as a travel single-dose (e.g., a grab-and-go disposable container).

When the supplemental spread 110 is contained within the canister 120, using the supplemental spread 110 may be like someone purchasing a tub of pre-workout or other supplement that may include instructions to take two scoops prior to workout. In the present disclosure, the instructions for using the supplemental spread 110 may instruct a user to mix, spread, or consume directly (e.g., prior to a workout) a specified amount (e.g., 2 scoops or 2 tablespoons). In some embodiments, the supplemental spread 110 may include children's vitamins, and the instructions may direct a user to spread the supplemental spread 110 over toast (e.g., a bread-based item 140).

Besides the canister 120 and the packet 130, other containers or methods may be used to store the supplemental spread 110. For example, the packet 130 may include but are not limited to the single-serve packet (e.g., a ketchup packet with a tearable edge) or a plastic-based container with a peel-away lid to expose the supplemental spread 110 in the container (e.g., a container similar to the single-serve jelly containers with peel-away top).

Storage benefits/general benefits: This spreadable feature of the supplemental spread 110 offers advantages (e.g., general storage stability) as compared to other form factors. For example, the supplemental spread 110 is more stable than pills, which can be crushed within single-packet packaging and generally require physical hand/skin contact for consumption. Powders can be messy and can spill.

Finally, with regards to the packaging itself, depending on the choice of storage container, the supplemental spread 110 can be stored with no oxygen exposure or sunlight exposure.

### Aspects

Various Aspects are described below. It is to be understood that any one or more of the features recited in the following Aspect(s) can be combined with any one or more other Aspect(s).

Aspect 1. A supplemental spread comprising: at least one of block chain amino acids (“BCAAs”), essential amino acids (“EAAs”), creatine, caffeine, taurine, tyrosine, alpha glycerylphosphorylcholine (alpha-GPC), or combinations thereof, wherein the supplemental spread has a viscosity ranging from about 450 centipoise to about 250,000 centipoise at a temperature ranging from about 20° C. to about 25° C.

Aspect 2. The supplemental spread of Aspect 1, wherein the supplemental spread comprises a spreadable form and is configured to be spread on a surface via a tool, whereby the supplemental spread sticks to the surface while being spread across the surface.

Aspect 3. The supplemental spread of Aspect 1 or Aspect 2, further comprising vitamin(s).

Aspect 4. The supplemental spread according to any of the preceding Aspects, further comprising an over-the-counter medicine(s) and/or a prescription medicine(s).

Aspect 5. The supplemental spread of Aspect 4, wherein the prescription medicine(s) comprise at least one of tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

Aspect 6. The supplemental spread of Aspect 4, wherein the over-the-counter medicine(s) comprise at least one of Ibuprofen, acetaminophen, dextromethorphan, pseudoephedrine, dimenhydrinate, diphenhydramine, or combinations thereof.

Aspect 7. The supplemental spread according to any of the preceding Aspects, wherein the supplemental spread is absent a protein source.

Aspect 8. The supplemental spread according to any of the preceding Aspects, wherein the supplemental spread is configured to be consumed on a stand-alone basis.

Aspect 9. The supplemental spread according to any of the preceding Aspects, wherein the supplemental spread is embedded in peanut butter, jam, or butter.

Aspect 10. A method of using a supplemental spread comprising: spreading the supplemental spread on a surface via a tool, whereby the supplemental spread sticks to the surface while being spread across the surface, wherein the supplemental spread comprises at least one of block chain amino acids (“BCAAs”), essential amino acids (“EAAs”), creatine, caffeine, taurine, tyrosine, alpha glycerylphosphorylcholine (alpha-GPC), or combinations thereof, and wherein the supplemental spread has a viscosity ranging from about 450 centipoise to about 250,000 centipoise at a temperature ranging from about 20° C. to about 25° C.

Aspect 11. The method of Aspect 10, wherein the supplemental spread further comprises vitamin(s).

Aspect 12. The method of Aspect 10 or Aspect 11, wherein the supplemental spread further comprises an over-the-counter medicine(s) and/or a prescription medicine(s).

Aspect 13. The method of Aspect 12, wherein the prescription medicine(s) comprise tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

Aspect 14. The method of Aspect 12, wherein the over-the-counter medicine(s) comprise at least one of Ibuprofen, acetaminophen, dextromethorphan, pseudoephedrine, dimenhydrinate, diphenhydramine, or combinations thereof.

Aspect 15. The method according to any of the preceding Aspects, wherein the supplemental spread is absent a protein source.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A supplemental spread comprising:
at least one of block chain amino acids (“BCAAs”), essential amino acids (“EAAs”), creatine, caffeine, taurine, tyrosine, alpha glycerylphosphorylcholine (alpha-GPC), or combinations thereof,
wherein the supplemental spread has a viscosity ranging from about 450 centipoise to about 250,000 centipoise at a temperature ranging from about 20° C. to about 25° C.

2. The supplemental spread of claim 1, wherein the supplemental spread comprises a spreadable form and is configured to be spread on a surface via a tool, whereby the supplemental spread sticks to the surface while being spread across the surface.

3. The supplemental spread of claim 1, further comprising vitamin(s).

4. The supplemental spread of claim 1, further comprising an over-the-counter medicine(s) and/or a prescription medicine(s).

5. The supplemental spread of claim 4, wherein the prescription medicine(s) comprise at least one of tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

6. The supplemental spread of claim 4, wherein the over-the-counter medicine(s) comprise at least one of Ibuprofen, acetaminophen, dextromethorphan, pseudoephedrine, dimenhydrinate, diphenhydramine, or combinations thereof.

7. The supplemental spread of claim 1, wherein the supplemental spread is absent a protein source.

8. The supplemental spread of claim 1, wherein the supplemental spread is configured to be consumed on a stand-alone basis.

9. The supplemental spread of claim 1, wherein the supplemental spread is embedded in peanut butter, jam, or butter.

10. A method of using a supplemental spread comprising:
spreading the supplemental spread on a surface via a tool, whereby the supplemental spread sticks to the surface while being spread across the surface,
wherein the supplemental spread comprises at least one of block chain amino acids (“BCAAs”), essential amino acids (“EAAs”), creatine, caffeine, taurine, tyrosine, alpha glycerylphosphorylcholine (alpha-GPC), or combinations thereof, and
wherein the supplemental spread has a viscosity ranging from about 450 centipoise to about 250,000 centipoise at a temperature ranging from about 20° C. to about 25° C.

11. The method of claim 10, wherein the supplemental spread further comprises vitamin(s).

12. The method of claim 10, wherein the supplemental spread further comprises an over-the-counter medicine(s) and/or a prescription medicine(s).

13. The method of claim 12, wherein the prescription medicine(s) comprise tofacitinib, upadacitinib, baricitinib, prednisone, meloxicam, naproxen, methotrexate, furosemide, torsemide, bumetanide, angiotensin-converting enzyme (ACE) inhibitors, furosemide, torsemide, bumetanide, spironolactone and eplerenone, cannabidiol, sildenafil, tadalafil, or combinations thereof.

14. The method of claim 12, wherein the over-the-counter medicine(s) comprise at least one of Ibuprofen, acetaminophen, dextromethorphan, pseudoephedrine, dimenhydrinate, diphenhydramine, or combinations thereof.

15. The method of claim 10, wherein the supplemental spread is absent a protein source.
